(19) United States
(12) Patent Application Publication
(10) Pub. No.: US 2018/0344887 A1
(43) Pub. Date: Dec. 6, 2018

(54) AUTOMATIC CLEANING DEVICE

(71) Applicant: BOBSON HYGIENE INTERNATIONAL INC., New Taipei City (TW)

(72) Inventors: Cheng-Feng CHEN, New Taipei City (TW); Cheng-Chang CHEN, New Taipei City (TW)

(21) Appl. No.: 15/614,114

(22) Filed: Jun. 5, 2017

(51) Int. Cl. A61L 2/24; E05B 1/00; G08B 21/18; B65D 83/26

(52) U.S. Cl. CPC A61L 2/24; E05B 1/0069; G01P 15/00; B65D 83/267; G08B 21/182

(57) ABSTRACT

An automatic cleaning device includes an accelerometer, a sprayer, a driving module and a processing unit. The accelerometer is mounted to a door, measures acceleration of the door, and outputs an acceleration value accordingly. The sprayer is for storing cleaner, and is operable to spray the cleaner. The processing unit is electrically connected to the driving module and the accelerometer, and determines whether the acceleration value is greater than an acceleration threshold, and generates a driving signal and transmits the same to the driving module for enabling the driving module to drive the sprayer to spray the cleaner when determining that the acceleration value is greater than the acceleration threshold.

### FIELD

The disclosure relates to an automatic cleaning device, more particularly to an automatic cleaning device for cleaning a door.

### BACKGROUND

Generally, a door handle that is disposed in a public space may not be cleaned or disinfected frequently. Bacteria may be bred on the door handle and may be spread by someone touching the door handle, which adversely affects public hygiene.

### SUMMARY

Therefore, an object of the present disclosure is to provide an automatic cleaning device for cleaning a door.

According to one aspect of the present disclosure, an automatic cleaning device includes an accelerometer, a sprayer, a driving module and a processing unit. The accelerometer is configured to be mounted to a door, to measure acceleration of the door, and to output an acceleration value according to the measurement of the acceleration of the door. The sprayer is for storing cleaner and is operable to spray the cleaner. The driving module is for driving the sprayer to spray the cleaner. The processing unit is electrically connected to the driving module and the accelerometer, and is configured to determine whether the acceleration value received from the accelerometer is greater than an acceleration threshold, and to generate a driving signal and transmit the driving signal to the driving module for enabling the driving module to drive the sprayer to spray the cleaner when determining that the acceleration value is greater than the acceleration threshold.

### BRIEF DESCRIPTION OF THE DRAWINGS

Other features and advantages of the present disclosure will become apparent in the following detailed description of the embodiments with reference to the accompanying drawings, of which:

FIG. 1 is a schematic block diagram illustrating an automatic cleaning device according to one embodiment of the present disclosure; and

FIG. 2 is a schematic view illustrating the automatic cleaning device being mounted to a door; and

FIG. 3 is a flow chart illustrating a method for cleaning the door implemented by the automatic cleaning device according to one embodiment of the present disclosure.

### DETAILED DESCRIPTION

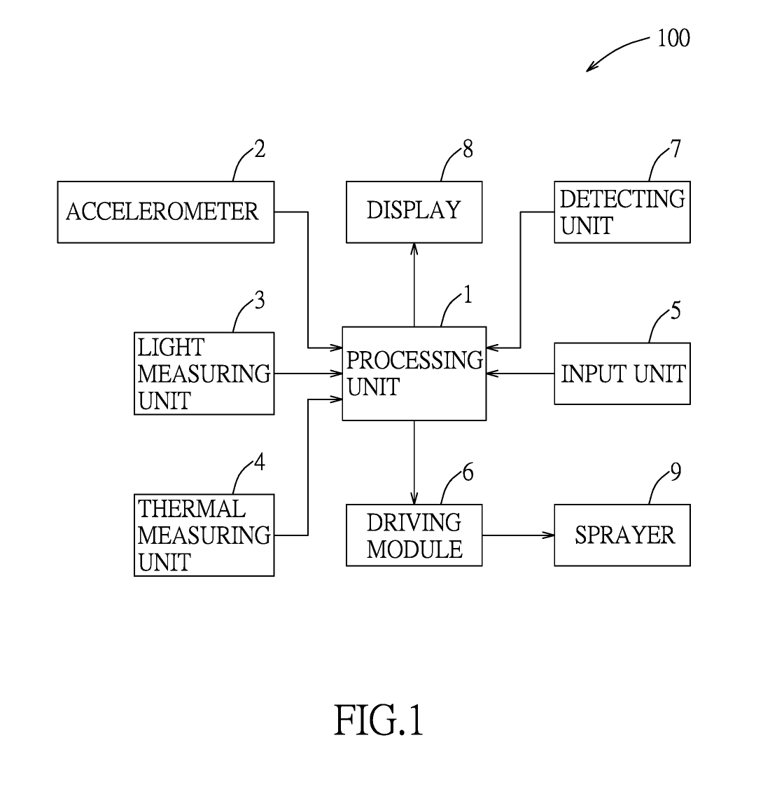

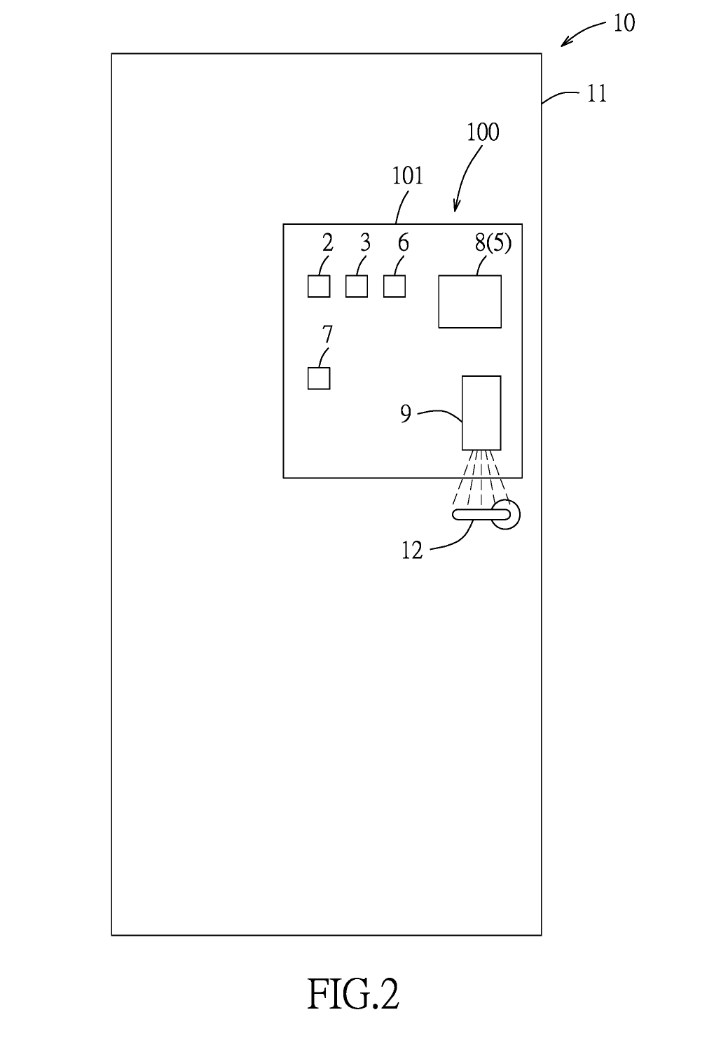

Referring to FIGS. 1 and 2, an automatic cleaning device 100 is mounted to a door 10 including a door panel 11 and a door handle 12 mounted on the door panel 11. The door handle 12 may be mounted to a surface of the door panel 11 that faces an indoor environment, e.g., a public lavatory. A user would rotate the door handle 12 to open the door 10 when leaving the public lavatory.

The automatic cleaning device 100 includes a processing unit 1, an accelerometer 2, a light measuring unit 3, a thermal sensing unit 4, an input unit 5, a driving module 6, a detecting unit 7, a display 8 and a sprayer 9.

The accelerometer 2 is mounted to the door panel 11 of the door 10 to measure acceleration of the door 10 and to output an acceleration value according to the measurement of the acceleration of the door 10, and is electrically connected to the processing unit 1 to transmit the acceleration value thereto.

The light measuring unit 3 is mounted to the door 10 for measuring ambient light at a place where the door 10 is disposed, is to generate an ambient luminance value according to the measurement of the ambient light and is electrically connected to the processing unit 1 for transmitting the ambient luminance value thereto. For example, the light measuring unit 3 is a light meter.

The thermal measuring unit 4 is mounted to the door panel 11 adjacent to the door handle 12 for measuring a temperature around the door handle 12, outputs a thermal signal to the processing unit 1 when the temperature around the door handle 12 is greater than a temperature threshold, and is electrically connected to the processing unit 1 for transmitting the thermal signal thereto. For example, the thermal measuring unit 4 is an infrared thermometer.

The processing unit 1 determines whether the acceleration value received from the accelerometer 2 is greater than an acceleration threshold and whether the ambient luminance value is greater than an luminance threshold. When the determinations made above are both affirmative, the processing unit 1 generates a driving signal for the first time and transmits the driving signal to the driving module 6 for enabling the driving module 6 to drive the sprayer 9 to spray the cleaner. In particular, the processing unit 1 starts timing a duration (e.g., by a timer) upon generating the driving signal, and further generates and transmits the driving signal again when the acceleration value is greater than the acceleration threshold, the ambient luminance value is greater than the luminance threshold, and the duration exceeds a time threshold. Note that the processing unit 1 holds back from generating the driving signal upon receipt of the thermal signal from the thermal sensing unit 4 since a user's hand may be on the handle 12 at that time. For example, the processing unit 1 may be a field-programmable gate array, an application-specific integrated circuit, or other integrated circuit capable of processing data.

The sprayer 9 is for storing cleaner, e.g., sanitizing fluid, is mounted to the door panel 11 close to the door handle 12, and is operable to spray the cleaner toward the door handle 12 as driven by the driving module 6. In other embodiments of the disclosure, the sprayer 9 may be mounted to other positions in the indoor environment, and may store air freshener to be sprayed in the air. The driving module 6 is electrically connected to the processing unit 1 for receiving the driving signal therefrom, and is configured to drive the sprayer 9 to spray the cleaner upon receiving the driving signal. For example, the driving module 6 is implemented by, but not limited to, a mechanical mechanism including a gear set for driving the sprayer 9, and a motor to be activated to drive the gear set upon receiving the driving signal.

The input unit 5 is electrically connected to the processing unit 1, and is configured to generate an initial value in response to user operation. The processing unit 1 stores an available value indicating a number of times that a remaining amount of the cleaner stored in the sprayer 9 can be sprayed out of the sprayer 9 (with each spraying action spraying a predefined amount of the cleaner), and the available value is set to equal the initial value upon receipt of the initial value from the input unit 5. For example, when a staff member operates the input unit 5 (e.g., by touching a button of or displayed on the input unit 5), the input unit 5 generates the initial value to equal a fixed default value of, e.g., five thousand indicating that the number of times the sprayer 9 is capable of spraying the cleaner is five thousand. In some embodiments, the input unit 5 is configured to allow user input of the initial value according to capacity of the sprayer 9, or an amount of refill of the cleaner.

In this embodiment, the input unit 5 is a touch panel integrated with the display 8 as a touch screen. In some embodiments, the input unit 5 may be implemented by a small keyboard or any suitable input interface accessible by the staff member, and the disclosure is not limited to this aspect.

The processing unit 1 decreases the available value by one every time when generating the driving signal, and generates an alarm signal when the available value equals zero.

The display 8 is electrically connected to the processing unit 1 for receiving the alarm signal, and displays an alarm message according to the alarm signal. The processing unit 1 controls the display unit 8 to display the available value and the initial value.

The detecting unit 7 is electrically connected to the processing unit 1, and generates a refill signal when the sprayer 9 is refilled with the cleaner. For example, the detecting unit 7 is a button disposed on an outer surface of the sprayer 9, and generates the refill signal upon being pressed. It should be noted that the detecting unit 7 may be hidden under a cover or be locked up to prevent unauthorized access to the detecting unit 7. In some embodiments, the detecting unit 7 is a button that is disposed within the sprayer 9 in contact with a container for containing the cleaner, and that is configured to generate the refill signal once the container is inserted into the sprayer 9 and contacts the button after being removed from the sprayer 9 for refill of the cleaner.

The processing unit 1 continuously generates the alarm signal until receipt of the refill signal from the detecting unit 7.

In this embodiment, the processing unit 1, the accelerometer 2, the light measuring unit 3, the thermal sensing unit 4, the input unit 5, the driving module 6, the detecting unit 7, the display 8 and the sprayer 9 are integrated in a housing 101 (see FIG. 2). Note that, in other embodiments of this disclosure, the components described above except for the light measuring unit 3 may be integrated in a housing, while the light measuring unit 3 is separated from the housing. Additionally, in other embodiments of this disclosure, the light measuring unit 3 may be omitted and the processing unit 1 generates and transmits the driving signal when the acceleration value is greater than the acceleration threshold and the duration exceeds a time threshold.

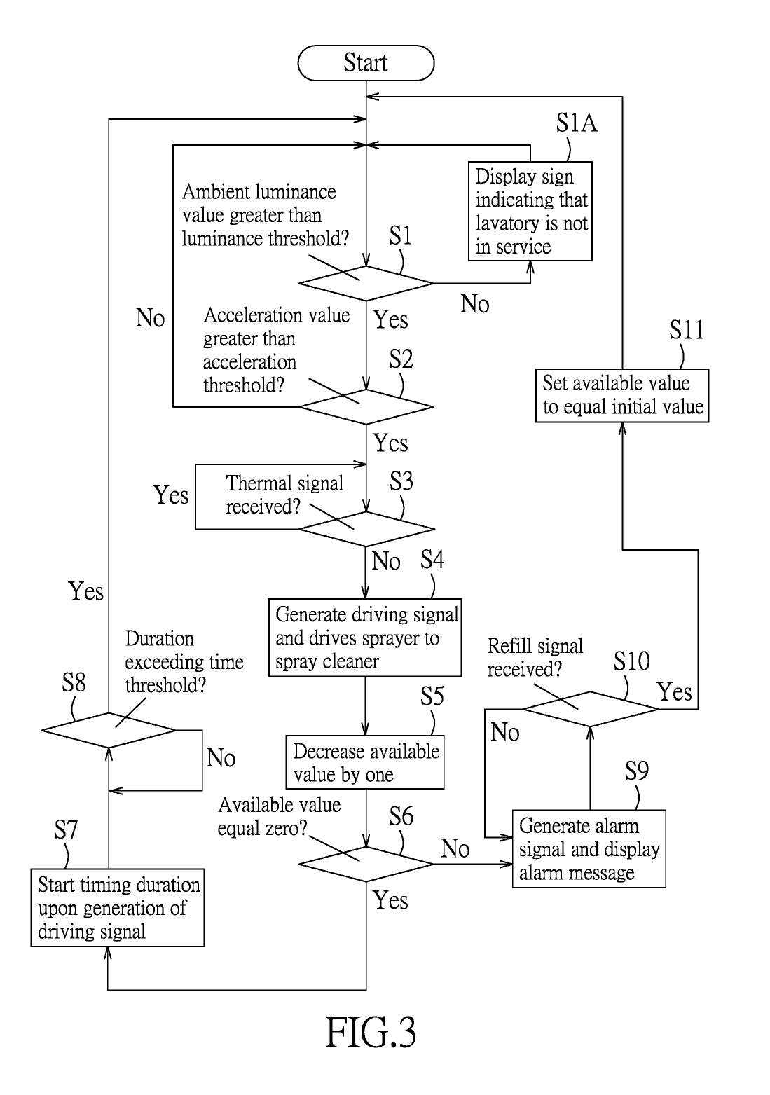

Referring to FIG. 3, a method for cleaning the door handle 12 of the door 11 to be implemented by the automatic cleaning device 100 according to one embodiment of the present disclosure is illustrated.

In step S1, the processing unit 1 determines whether the ambient luminance value generated by and transmitted from the light measuring unit 3 is greater than the luminance threshold. When the determination made in step S1 is affirmative, the flow of the method goes to step S2; otherwise, the flow goes to step S1A. For example, when the ambient luminance value is greater than the luminance threshold, lighting fixtures disposed in, e.g., the public lavatory, where the door 10 is disposed is turned on and the public lavatory is in service hours. When the determination made above is negative, the display 8 displays, in step S1A, a sign indicating that the public lavatory is not in service hours, and the flow returns to step S1.

Note that before step S1, the staff member has to operate the input unit 5 to generate the initial value and the processing unit 1 sets the available value to equal the initial value for activating the automatic cleaning device 100 for the first time. In some embodiments, the available value is set to equal a predetermined value when the automatic cleaning device 100 is manufactured.

In step S2, the processing unit 1 determines whether the acceleration value measured by the accelerometer 2 is greater than the acceleration threshold. When the determination made in step S2 is affirmative, the flow goes to step S3; otherwise, the flow returns to step S1. When the acceleration value is greater than the acceleration threshold, it means that the door 10 may have lust been moved by a user.

In step S3, the processing unit 1 determines whether the thermal signal generated by and transmitted from the thermal measuring unit 4 is received. The thermal measuring unit 4 generates and transmits the thermal signal upon detecting that the temperature around the door handle 12 is greater than the temperature threshold, and the increase in the temperature around the door handle 12 from the temperature threshold may be attributed to the user's hand recently staying on the door handle 12. When the determination made in step S3 is negative, the flow goes to step S4; otherwise, the flow goes back to step S3.

In step S4, the processing unit 1 generates the driving signal to be received by the driving module 6 such that the driving module 6 drives the sprayer 9 to spray the cleaner toward the door handle 12 in response to receipt of the driving signal. By virtue of step S3, the driving module 6 will not drive the sprayer 9 to spray the cleaner when the temperature around the door handle 12 has risen above the temperature threshold, avoiding accidentally spraying the cleaner on the user's hand. In some embodiments, the thermal measuring unit 4 is configured to determine whether the temperature measured thereby is greater than the room temperature, which serves as the temperature threshold, to generate the thermal signal when the measured temperature is greater than the room temperature, and to stop generating the thermal signal when the measured temperature is substantially equal to the room temperature. The room temperature can be constantly or periodically monitored by the automatic cleaning device 100.

Subsequent to step S4, in step S5, the processing unit 1 decreases the available value by one. In step S6, the processing unit 1 determines whether the available value equals zero. When the determination made in step S6 is affirmative, the flow goes to step S7; otherwise, the flow goes step S9.

In step S7, the processing unit 1 starts timing a duration upon generation of the driving signal. Subsequently, in step S8, the processing unit determines whether the duration exceeds the time threshold. When the determination made in step S8 is affirmative, the flow goes back to step S1; otherwise, the flow returns to step S8. Accordingly, the processing unit 1 will not generate the driving signal again within a duration not exceeding the time threshold.

In step S9, the processing unit 1 generates the alarm signal to be received by the display 8 that displays the alarm message thereon according to the alarm signal. In step S10, the processing unit 1 determines whether the refill signal generated by and received from the detecting unit 7 is received. When the determination made in step S10 is affirmative the flow goes to step S11; otherwise, the flow returns step S9.

In step S11, the processing unit 1 sets the available value to equal the predetermined value and the flow goes back to step S1.

To sum up, in the present disclosure, by virtue of the light measuring unit 3, the automatic cleaning device 100 can determine whether the public lavatory, for instance, is in service hours. Further, by virtue of the accelerometer 2, the sprayer 9 may spray the cleaner on the door handle 12 right after the user opens the door 10. In addition, by virtue of timing the duration in step S7, the sprayer 9 only sprays the cleaner again after at least a predefined interval (i.e., the time threshold), avoiding excessively frequent spraying of the cleaner and allowing the driving of the sprayer 9 to spray the cleaner to be conducted in a relatively effective and also economical manner. Moreover, the thermal measuring unit 4 transmits the thermal signal to the processing unit 1 to prevent the processing unit 1 from generating the driving signal when the measured temperature is greater than the temperature threshold, which is for example the room temperature, avoiding accidentally spraying the cleaner on the user's hand.

In the description above, for the purposes of explanation, numerous specific details have been set forth in order to provide a thorough understanding of the embodiment. It will be apparent, however, to one skilled in the art, that one or more other embodiments maybe practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. An automatic cleaning device comprising:
an accelerometer configured to be mounted to a door, to measure acceleration of the door, and to output an acceleration value according to the measurement of the acceleration of the door;
a sprayer for storing cleaner and operable to spray the cleaner;
a driving module for driving said sprayer to spray the cleaner; and
a processing unit electrically connected to said driving module and said accelerometer, and configured to determine whether the acceleration value received from said accelerometer is greater than an acceleration threshold, and to generate a driving signal and transmit the driving signal to said driving module for enabling said driving module to drive said sprayer to spray the cleaner when determining that the acceleration value is greater than the acceleration threshold.

2. The automatic cleaning device as claimed in claim 1, wherein said processing unit is further configured to start timing a duration upon generating the driving signal, and to generate the driving signal again when it is determined that the acceleration value is greater than the acceleration threshold and the duration exceeds a time threshold.

3. The automatic cleaning device as claimed in claim 2, further comprising a light measuring unit configured to be mounted to the door for measuring ambient light at a place where the door is disposed, and to generate an ambient luminance value according to the measurement of the ambient light,
wherein said processing unit is further electrically connected to said light measuring unit for receiving the ambient luminance value therefrom, and is configured to generate the driving signal when the acceleration value is greater than the acceleration threshold, the duration exceeds the time threshold, and the ambient luminance value is greater than an luminance threshold.

4. The automatic cleaning device as claimed in claim 1, further comprising a light measuring unit configured to be mounted to the door for measuring ambient light at a place where the door is disposed, and to generate an ambient luminance value according to the measurement of the ambient light,
wherein said processing unit is further electrically connected to said light measuring unit for receiving the ambient luminance value therefrom, and is configured to generate the driving signal when the acceleration value is greater the acceleration threshold and the ambient luminance value is greater than an luminance threshold.

5. The automatic cleaning device as claimed in Claim further comprising an input unit electrically connected to said processing unit, and configured to generate an initial value in response to user operation,
wherein said processing unit stores an available value indicating a number of times that a remaining amount of the cleaner stored in said sprayer is to be sprayed out of said sprayer, and the available value is set to equal the initial value upon receipt of the initial value from said input unit.

6. The automatic cleaning device as claimed in claim 5, wherein said processing unit is further configured to decrease the available value by one every time when generating the driving signal, and to generate an alarm signal when the available value equals zero.

7. The automatic cleaning device as claimed in claim 6, further comprising a display that is electrically connected to said processing unit for receiving the alarm signal and that displays an alarm message according to the alarm signal,
wherein said processing unit is further configured to control said display to display the available value and the initial value.

8. The automatic cleaning device as claimed in claim 6, further comprising a detecting unit electrically connected to said processing unit, and configured to generate a refill signal when detecting that said sprayer is refilled with the cleaner,
wherein said processing unit is configured to continuously generate the alarm signal until receipt of the refill signal from said detecting unit.

9. The automatic cleaning device as claimed in claim 1, the door including a door handle, the cleaner stored in said sprayer being a sanitizing fluid,
wherein said sprayer is configured to be mounted to the door close to the door handle and to spray the sanitizing fluid toward the door handle.

10. The automatic cleaning device as claimed in claim 9, further comprising a thermal measuring unit that is configured to be mounted to the door adjacent to the door handle for measuring a temperature around the door handle, that is electrically connected to said processing unit, and that is configured to output a thermal signal to said processing unit when the temperature around the door handle is greater than a temperature threshold,
wherein said processing unit holds back from generating the driving signal upon receipt of the thermal signal from said thermal measuring unit.
